(19) United States
(12) Patent Application Publication
(10) Pub. No.: US 2023/0218199 A1
(43) Pub. Date: Jul. 13, 2023

(54) A SYSTEM FOR ASSESSING HUMAN MOVEMENT AND BALANCE

(71) Applicant: University of Miami, Miami, FL (US)

(72) Inventors: Kyoung Jae Kim, Miami, FL (US); Robert Gailey, Miami, FL (US); Ignacio Gaunaud, Miami, FL (US); Christopher Bennett, Miami, FL (US)

(73) Assignee: University of Miami, Miami, FL (US)

(21) Appl. No.: 17/995,548

(22) PCT Filed: Apr. 9, 2021
(86) PCT No.: PCT/US2021/026589

§ 371 Date: Oct. 5, 2022

Related U.S. Application Data
(60) Provisional application No. 63/008,248, filed on Apr. 10, 2020.

(51) Int. Cl. A61B 5/11; A61B 5/00

(52) U.S. Cl. CPC A61B 5/1114; A61B 5/4023; A61B 5/7221; A61B 5/0024; A61B 2505/09; A61B 2503/10

(57) ABSTRACT

Systems and methods for assessing, monitoring, or theranosing a condition or disorder based on a comparison of limb stability for one or more limbs of a subject from a baseline. The method includes placing two or more inertial measurement sensors on the limbs of the subject, acquiring baseline limb excursion data from the inertial measurement sensors while a patient is performing at least one of a static balance activity and a dynamic balance activity by tracking the relative displacement of the respective two or more inertial measurement sensors; acquiring post-injury limb excursion data after an injury from the inertial measurement sensors while a patient is performing at least one of a static balance activity and a dynamic balance activity; and determining the activity clearance index as a function of a comparison of the baseline limb excursion data compared to the post-injury limb excursion data.

### CROSS REFERENCE TO RELATED APPLICATION

This application claims the benefit of, and priority from, U.S. Provisional Application No. 63/008,248, entitled “A MOBILE WIRELESS SENSOR SYSTEM FOR ASSESSING HUMAN MOVEMENT AND BALANCE,” filed Apr. 10, 2020, the entirety of which is incorporated by reference herein.

### BACKGROUND OF THE INVENTION

This invention generally relates to systems and methods described herein can be used for assessing human movement, balance, and other health issues.

People in the general population, the elderly, patients, athletes and who have a musculoskeletal weakness, neuromuscular disease, sustained an injury to the lower limb, or have had a head injury often will demonstrate loss of balance during static and dynamic activities, in addition to a compromised ability to perform agility skills. Current medical protocols do not take into account these important variables in diagnosing such ailments. For example, 1) quantitative measures of balance and agility of limbs, including assessing the excursion area and the differences between, for example, the shank (lower leg) and thigh; 2) upper limb motions related to sports such as throwing, swing and blocking activities and everyday functional tasks such as reaching or overhead activities can be quantified; 3) spinal motions during different postures (standing, kneeling and sitting) or tasks (lifting objects, walking or work related activities) can be quantified with multiple metrics, none of which exist. Moreover, any existing traditional measure of balance and agility requires either 1) expensive equipment, is confined to a laboratory and requires time for quantitative analysis, or 2) uses low cost equipment, can be performed in most environments but is restricted to qualitative analysis and subjective observation. The prior art does not provide for a low cost and quantitative assessments of human motion related to specific anatomical structures. Moreover, while certain prior art systems and methods may observe human motion for diagnostics, these systems often compare data against a general population, or a baseline goal. This type of analysis fails to consider the importance of comparative analysis of a single patient's baseline against the patient's current performance, as well as a comparison between left and right sided performances.

Therefore, there is a particular need for a low cost mobile device that uses relatively low-cost equipment, in any environment and provides quantitative assessment of human motion related to specific anatomical structures of the lower limb during both static and dynamic conditions.

### SUMMARY OF THE INVENTION

The present invention is designed for assessing health conditions as a function of static and dynamic limb movement. For example, the present invention can be used for assessing human balance and movement at the intrinsic level of the lower limb to determine current balance and functional capabilities, determine the risk of injury, quantify restoration of balance and movement during rehabilitation, assist with exercise and treatment prescription, enhance performance and to reduce the risk injuries (examples include, but are not limited to, falls, overuse injuries, degenerative joint diseases, and non-contact athletic injuries). The present invention can make use of assessments during static balance activities of single limb stance, quiet standing on two lower limbs on any surface, non-compliant, compliant, or dynamic. Additionally, or alternatively, the instant system and methods can make use of dynamic agility activities, in all planes of movement including forwards, backwards, sideways and in a zig-zag pattern during an assessment. Further, the instant system and methods can make use of upper limb motions, including but not limited to, isolated motions in cardinal planes or function motions reaching, throwing, lifting, and carrying and/or spinal motions, including but not limited to, flexion, extension, side bending, rotation, and functional task lifting or sports movement.

The systems and methods of the present invention can be used, for example, for the following populations: 1) the general population who require balance or movement assessment; 2) elderly people with impaired balance or motor control due to the normal aging process, illness, or injury; 3) patients with a medical history associated with balance, motor control, posture or agility, injury to the lower limb and other related medical conditions; 4) people with degenerative diseases such as, but not limited to, neuropathy, arthritis, or other systemic conditions; 5) athletes (recreational and elite) who wish to have required preseason or performance assessment, have sustained an injury, seeking injury prevention or performance enhancement treatment; and 6) military physical screening for risk of injury prior to physical or specialized training and military readiness.

The systems and methods can utilize the following modalities for injury prevention, treatment, and performance enhancement: 1) numeric summary of balance and movement capabilities, graphic illustrations, animated motion graphics, categorical data analysis with color-coded, and/or images for performance and injury risk ranking; 2) prescription of targeted exercise and training programs based on limitations in static balance and dynamic agility activities; 3) real-time biofeedback (auditory, visual or haptic stimulus) from a sensor system for treatment and performance enhancement of balance and movement strategies.

One aspect of the instant system and methods can involve quantification of human motion and balance strategies, deviations associated with age, weakness, neuromuscular injuries or disease and concussive injuries during static balance and dynamic agility activities. Embodiments of the present invention can include novel algorithms for quantifying balance strategies and deviations during standing and various movements using inertial measurements.

In some embodiments, the system and method can use a mobile wireless sensor system for the assessment of human motion and balance using two novel measures. For the sake of ease, the discussion will be made with respect to a patient's lower limbs, though the sensors and data can be directed to other limbs and anatomy as noted above. The data collection system can include a plurality of nine (9) degree of freedom Inertial Measurement Unit (IMU) sensor, comprising an accelerometer, gyroscope, and magnetometer. While the application makes use of the term IMU, it is noted that other such motion capture sensors can be used in place of an IMU and are understood to be comparable. These IMU sensors can be placed on a user's left and right thigh and left and right shank. The IMU sensors, or mobile wireless sensor system can be used for the assessment of human motion and balance using two novel measures. These measures are defined as the region of limb stability (ROLS) and the transitional angular displacement of segments (TADS) that assesses the responsiveness of the thigh and shank and the resultant motion of the anatomical joints of the lower limb during standing, walking, running, multi-directional movements, sports, and other mobility activities. The ROLS is defined as a measure of the observed region of knee excursion, defined by thigh and shank movements on the horizontal plane during single lower limb stance (SLS) on the left and right lower limbs. These excursions are represented on a polar plot with the medio-lateral (M-L) displacement and the anterior-posterior (A-P) displacement of the thigh and shank. The TADS can be defined as being computed from sagittal angular velocities of the shank IMU during the transition periods of the Four Meter side step test (FMSST). The transition periods quantify changes of direction from side to side, including knee joint angle, speed, and quality of the motion. TADS can measure postural changes between segments (thigh and shank) or within a joint in the sagittal, frontal, and transverse plane.

The ROLS and TADS are measures designed to: 1) establish baseline balance and mobility symmetry between lower limbs, assess post-injury healing assisting clinicians, coaches and athletes with return-to-play decisions; 2) predict balance or mobility asymmetries between limbs for the prediction of risk injury; 3) identify movement limitations related to balance and mobility for exercise prescription to promote evidence-based rehabilitation; 4) determine change over time after neuromuscular injuries or disease and concussive or general brain injuries to determine thresholds appropriate for return to activity, return to work, return to practice, return to duty, return to independence, and return to sport; and/or 5) assess balance and mobility capabilities related to sport or fitness, provide quantitative analysis, feedback, for performance enhancement training.

The system and methods of the present invention overcomes the disadvantages of the prior art by providing a low cost mobile device that uses relatively low-cost equipment, in any environment and provides quantitative assessment of human motion related to specific anatomical structures of the lower limb during both static and dynamic conditions

### BRIEF DESCRIPTION OF THE DRAWING FIGURES

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1 is a schematic diagram of a system according to an embodiment;

FIG. 2A is a front elevation view of sensor alignment according to an embodiment;

FIG. 2B is a top view of the sensor alignment of FIG. 2A;

FIG. 3 is a side view of a single limb stance test;

FIGS. 4A, 4B, and 4C are graphical representations of the measurements from the single limb stance test of FIG. 3;

FIG. 5 is a table illustrating various metrics of five athletes who sustained injuries;

FIG. 6 is a table illustrating descriptive statistics;

FIG. 7 is a table illustrating descriptive values for ROLS and ROLS between limbs for those non-injured and injured subjects;

FIG. 8A-C illustrate graphs showing an uninjured patient's ROLS metrics;

FIGS. 9A-9C illustrate graphs showing a patient's ROLS metrics having right medial collarteral ligament injury;

FIGS. 10A-C illustrate graphs showing a patient's ROLS metrics having a right anterior cruciate ligament injury;

FIGS. 11A and 11B illustrate graphs showing ROLS mean excursion diagrams;

FIGS. 12A and 12B illustrate the ROLS post-concussive excursion profile;

FIG. 13 is a table showing differences in healthy/pre-concussion and post-concussion study values;

FIG. 14 is a table showing ROLS post-concussive excursion index;

FIGS. 15A and 15B are ROLS Mean Excursion Diagrams;

FIG. 16 is a table illustrating a comparison between sacrum sway area and ROLS;

FIG. 17 is a schematic view of the four meter side step test;

FIG. 18A is a graph illustrating the sagittal angular velocities recorded during the FMSST;

FIG. 18B is a graph illustrating the frontal angular velocities recorded during the FMSST;

FIG. 18C is a graph illustrating coupled shank angular displacements;

FIG. 18D is a graph showing an enlarged view of one directional transition and area under the wave during knee flexion and extension;

FIGS. 18E and 18F show a graphical representation of the knee flexion and extension shown in FIG. 18D;

FIG. 19 is a table showing results of TADS and FMSST times and test-retest reliability;

FIG. 20 is a table showing the injury risk index with musculoskeletal injury prediction;

FIG. 21 is a table showing the ROLS post-concussive excursion index;

FIG. 22 is a table showing FMSST times and TADS Symmetry Index (SI); and

FIG. 23 is a table showing results of the tests related to the FMSST and TADS SI.

### DESCRIPTION OF THE INVENTION

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal. A person skilled in the art will recognize that various other medical condition other than those explicitly named herein can be diagnosed with the instant systems and methods. While reference is made to lower limbs of a subject, one of ordinary skill in the art will understand that the instant methods and system can be applied to other anatomy of a subject as well. While reference is made to the term inertial measurement units (IMU), it is noted that other such wearable motion capture sensors, or inertial measurement sensors, can be used in place of an IMU and are understood to be comparable. While reference is made to a human subject any living subject is understood be within the scope of this disclosure.

In accordance with an embodiment of the present invention, a new and novel system and method for assessing health conditions is provided. The present invention can be used for assessing health conditions as a function of static and dynamic limb movement. For example, as will be discussed in detail below, the present invention can be used for assessing balance and movement at the intrinsic level of the lower limb to determine current balance and functional capabilities, determine the risk of injury, quantify restoration of balance and movement during rehabilitation, assist with exercise and treatment prescription, enhance performance and to reduce the risk injuries (examples include falls, overuse injuries, and non-contact athletic injuries). The present methods and systems method for assessing, monitoring, or theranosing a condition or disorder based on an assessment of the subject's comparative balance. The present invention can make use of assessments during static balance activities of single limb stance, quiet standing on two lower limbs on any surface, non-compliant, compliant, or dynamic. Additionally, or alternatively, the instant system and methods can make use of dynamic agility activities, in all planes of movement including forwards, backwards, sideways and in a zig-zag pattern during an assessment. The static and dynamic activities, the resulting data, and the implications of such data will now be discussed in detail.

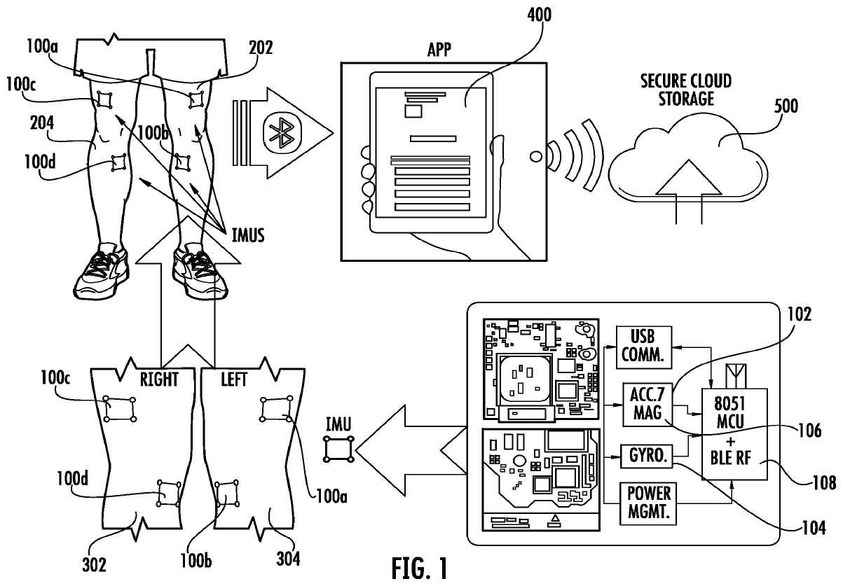

In a first embodiment, as shown in FIG. 1, a mobile wireless sensor system for the assessment of human motion and balance is provided. The mobile wireless sensor system 100 can generally include four sensors and can be an inertial measurement unit (IMU) sensors. Alternatively, more, or less sensors may be used depending on the limb or portion of the human anatomy that is being observed. The sensor of the data collection system can include a 9 degree of freedom Inertial Measurement Unit (IMU) sensor 100 comprising an accelerometer 102, gyroscope 104, and magnetometer 106. The IMU sensor 100 can additionally include a communication module, e.g. BLUETOOTH, to communicate with a digital data device 400 which can, in turn, upload data to a secure cloud storage 500. As shown, a first sensor 100a can be placed on the thigh of the left leg 202, a second sensor 100b can be placed on the shank of the left leg 202, a third sensor 100c can be placed on the thigh of the right leg 204, and a fourth sensor 100d can be placed on the shank of the right leg 204. The system and methods herein take data from the sensors to determine two novel measures: the region of limb stability (ROLS) and the transitional angular displacement of segments (TADS) that can assess the responsiveness of the thigh and shank and the resultant motion of the anatomical joints of the lower limb during standing, walking, running, multi-directional movements, sports, and other mobility activities.

The ROLS and TADS are measures that are designed to 1) establish a baseline balance and mobility symmetry between lower limbs, assess post-injury healing levels that can assist clinicians, coaches, and athletes with return-to-play decisions; 2) predict balance or mobility asymmetries between limbs for the prediction of risk injury; 3) identify movement limitations related to balance and mobility for exercise prescription to promote evidence-based rehabilitation; 3) determine change over time after neuromuscular injuries or disease and concussive injuries to determine thresholds appropriate for return to activity, return to work, return to practice and return to sport; 4) assess balance and mobility capabilities related to sport or fitness, provide quantitative analysis, feedback, for performance enhancement training.

The instant system can use the sensors to determine the ROLS and TADS measures which can be computed using acceleration and angular velocity data from IMUs. Two examples of use with the system can include:

1) the ROLS is a measure of the observed region of knee excursion, defined by thigh and shank movements on the horizontal plane during single lower limb stance (SLS) on the left and right lower limbs. These excursions are represented on a polar plot with the medio-lateral (M-L) displacement and the anterior-posterior (A-P) displacement of the thigh and shank.

2) The TADS can be computed from sagittal angular velocities of the shank IMU during the transition periods of a Four Meter Side Step Test (FMSST). These transition periods can quantify changes of direction from side to side, including knee joint angle, speed, and quality of the motion. Thus, the TADS can measure postural changes between segments (thigh and shank) or within a joint in the sagittal, frontal, and transverse plane.

Following data collection with IMUs, the system can complete the following steps: A) virtual sensor alignment; B) quantification of static balance activities using the ROLS metric; C) determination of the reliability of the ROLS metric using a single limb stance (SLS) activity; D) calculation, quantification, and display of the ROLS metric; E) a comparison of the ROLS to similar metrics and technologies; F) a quantification of dynamic balance activities and movement transition using the TADS metric; G) determination of the reliability of the TADS metric; and H) calculation, quantification, and display of the TADS metric. Each of which will now be discussed in turn.

The correct placement of the IMU relative to the cardinal directions of the body, anterior-posterior (A-P), superior-inferior (S-I), and medial-lateral (M-L), is necessary to accurately capture joint motions with the IMU. To reduce error, soft tissue and boney landmarks that were relatively flat and had minimal motion during activity can be identified. Of note, errors can be reported with IMU placement, those errors can include inconsistency of placement between subjects, artifactual displacements due to soft-tissue deformations, regions of instability where IMUs do not rest flush against the subject's body, and anatomical differences in the curvature of the musculature of the lower limbs across individuals. Each of these sources of error are addressed herein using a combination of manual and computational alignment procedures.

For example, soft tissue artifacts and IMU instability can be minimized by using a stretchable knee sleeve 302, 304 fitted with small elastic bands that keep the sensors in place, i.e., approximately 4 cm below the knee joint line at the medial tibial flare on the shank and approximately 4 cm above the knee joint line, along with the distal iliotibial band on the thigh. The need to avoid placement of the IMUs over soft tissue and contracting muscles limits potential sites for placement of IMUs on the lower limbs. One potential site for the placement of the IMU can include the medial tibial flare, a flat surface lacking muscle, for example as a suitable site for the distal shank sensor. One example of a proximal thigh site for IMU placement is just anterior to the iliotibial band insertion distal to the muscle belly of the vastus lateralis, the most lateral aspect of the quadriceps muscle. This location on the thigh can prevent any disruption of normal patellar tracking. The lateral placement can prevent the IMU from disrupting the limbs normal adducted positioning during swing and stance phases of gait. The use of the elastic knee sleeve allows for efficient donning and doffing of the IMU's and can provide a consistent location of the sensors on the lower limb, to minimize user error during placement of the IMU. Alternatively, or additionally, the IMUs can be fixed to a subject by other means such as, but not limited to, an adhesive, double sided tape, or other wraps that can wrap around the limb. Further, additional IMUs can be placed on a center of the sacrum of a subject for additional data.

While the aforementioned placement locations can provide consistent landmarks between people with relatively flat and uniform anatomy, they are neither aligned with each other nor aligned with a generalizable reference frame. Therefore, a two-phase signal processing alignment procedure can be implemented to establish a known relationship between the four relevant reference frames: the sensor frame S=<Sₓ, S_{y}, S_{z}>, the joint frame J=<Jₓ, J_{y}, J_{z}>, the body frame B=<Bₓ, B_{y}, B_{z}>, and the global frame G=<Gₓ, G_{y}, G_{z}>.

The fixed, time-invariant rotation from S to J, R_{StoJ}, can be computed by an alignment algorithm consisting of a Gauss-Newton minimizer that searches for the set of joint axes j_{Shank} and j_{Thigh} to minimize the cost function in (1), where ω_{Shank} and ω_{Thigh} were 3×N matrices containing the measured angular velocities of the shank and thigh, respectively over N time points.

∥ω_{Shank}(t)×j_{Shank}∥₂−∥ω_{Thigh}×j_{Thigh}∥₂=0,∀t (1)

The Gauss-Newton minimizer can operate under the hinged joint assumption that off-axis motion is observed equally by both the thigh IMU and the shank IMU. The difference between cross products of j and ω for the shank and thigh can be assumed to be zero. The minimizer computes j^{S} which is the joint axis in S, where

j^{S}= [symbol] jₓ^{S},j_{y}^{S},j_{z}^{S} [symbol] (2)\

and where j^{J}, the joint axis in J, was defined as the unit vector

j^{J}= [symbol] 1,0,0 [symbol] . (3)

The matrix R_{StoJ} then corresponded to the rotation between j^{J} and j^{S}

j^{J}=R_{StoJ}j^{S} (4)

that mapped S onto J.

The time-variant rotation between J and B, R_{JtoB}, can be computed through the use of a Mahony filter. A Mahony filter can be defined as a computationally inexpensive form of complementary filter robust to heading errors. It can establish the body reference frame B, with respect to gravity, and can track the changes in sensor orientation relative to that initial reference frame, B⁰.

Finally, R_{BtoG}, the rotation from B to G can be determined. For single lower limb stance (SLS) activities, for example, B can be assumed to be time-invariant and equal to G since both tasks require that the person to stay facing straight forward at all times, t; therefore, R_{BtoG} can be assumed to be equal to the identity matrix I. The complete set of rotations to transform from the sensor frame S to the global frame G is

G=R_{BtoG}B=IR_{JtoB}(t)J=R_{JtoB}(t)R_{StoJ}S. (5)

The sensor frame S can be specified by the physical axes of the IMU. The joint frame J can be defined as having one axis aligned with the knee joint, while the other two axes remained unconstrained to an initial position. The body frame B can be defined in the SI axis by the gravity vector g, in the ML axis by the projection of the joint axis onto the plane perpendicular to gravity, j·g, and in the AP axis by the cross product of g and j·g, such that

$$B = [ \begin{matrix}{B}_{x} \\ {B}_{y} \\ {B}_{z}\end{matrix} ] = [ \begin{matrix}g \\ j \cdot g \\ j \times ( j \cdot g )\end{matrix} ] = [ \begin{matrix}SI \\ ML \\ AP\end{matrix} ] . \qquad ( 6 )$$

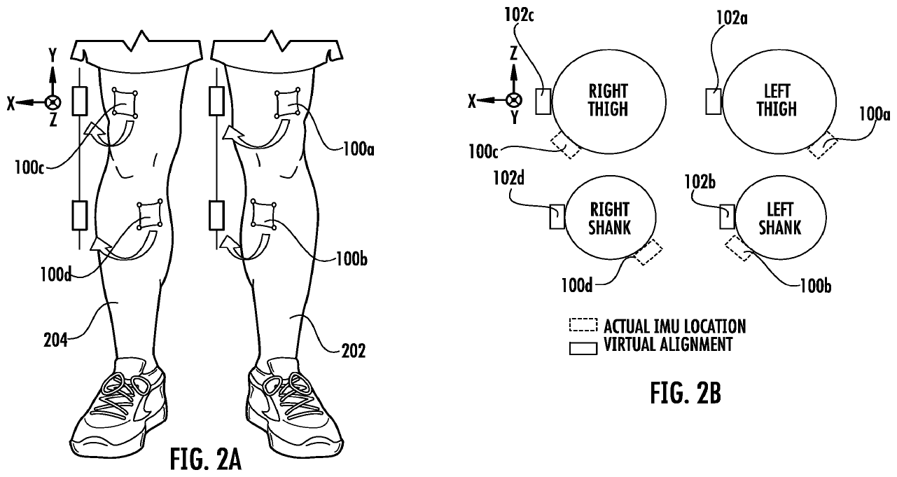

The global frame G axes are defined as the initial position of B⁰ while the person remains still in an upright standing position, as shown in FIGS. 2A and 2B.

Alternatively, the following algorithm can be used for calculating R_{StoJ}, and thus the alignment of the sensors. The subject can be directed to first perform a stand still step for a few seconds, followed by a linear gait step for a few steps. The fixed, time-invariant rotation from the sensor frame of reference, S to the knee joint frame of reference J, can be captured in the rotation matrix R_{StoJ}. R_{StoJ} can be computed by an alignment algorithm that estimates the sensors' roll, pitch, and yaw compared to that of the knee joint, J. This algorithm first creates a rotation matrix that corrects for roll and pitch using the sensors' acceleration values α=<αₓ, α_{y}, α_{z}> captured during a stand-still period.

$${R}_{a} = [ \begin{matrix}\frac{{a}_{y}}{‖ {a}_{xy} ‖} & \frac{- {a}_{x}}{‖ {a}_{xy} ‖} & 0 \\ \frac{{a}_{x}}{‖ {a}_{xy} ‖} & \frac{{a}_{y}}{‖ {a}_{xy} ‖} \cdot \frac{{a}_{y}}{‖ {a}_{yz} ‖} & \frac{{a}_{z}}{‖ {a}_{yz} ‖} \\ 0 & \frac{- {a}_{z}}{‖ {a}_{yz} ‖} & \frac{{a}_{y}}{‖ {a}_{yz} ‖}\end{matrix} ]$$

Where ∥α_{yz}∥=\sqrt{α_{y}²+α_{z}²} and ∥α_{xy}∥=\sqrt{αₓ²+α_{y}²}

Then this algorithm can create a rotation matrix that corrects for yaw using the sensors' gyroscope values ω=<ωₓ, ω_{y}, ω_{z}> captured during a few steps of linear gait.

$${R}_{ω} = [ \begin{matrix}\frac{{ω}_{x}}{‖ {ω}_{xz} ‖} & 0 & \frac{{ω}_{z}}{‖ {ω}_{xz} ‖} \\ 0 & 1 & 0 \\ \frac{- {ω}_{z}}{‖ {ω}_{xz} ‖} & 0 & \frac{{ω}_{x}}{‖ {ω}_{xz} ‖}\end{matrix} ]$$

Where ∥ω_{xz}∥=\sqrt{ωₓ²+ω_{z}²}

Finally, the two rotation matrices are combined into a single rotation matrix for mapping the sensors' frame of reference, S, onto the knee joint frame of reference, J.

R_{StoJ}=Rω·R_{α}

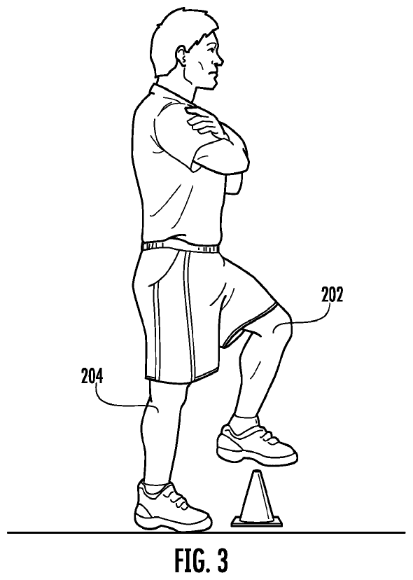

Once the alignment of the sensor frame to the joint frame calculation is complete, it is then possible to have the user perform the static balance activities for quantification using the region of limb stability (ROLS) metric. The ROLS metric is a novel measure to quantify the stability of the lower-limb segments (i.e., thigh and shank) during static balance activities of single limb stance (SLS), as shown in FIG. 3, and quite standing on both lower limbs. In general, the SLS test can include a participant who must stand unassisted on one leg 204 and is timed in seconds from the time one foot 202 is flexed off the floor to the time when it touches the ground or the standing leg or an arm leaves the hips. ROLS is a measure of bi-directional thigh and shank segment excursions in the horizontal plane and can be computed using acceleration data from two IMUs, placed approximately 6.35 cm (2.5 inches) proximal and distal to the knee joint line. These excursions are represented on a polar plot with the x-axis representing M-L displacement, the y-axis representing AP displacement. These computations can be of a traced path in the horizontal plane using a tri-axial accelerometer-based system. Segment excursions can be used to compute the ROLS metric by tracking the relative displacement of the shank and thigh IMUs as determined from double integration of the acceleration in B. Acceleration in B, α^{B}(t), was determined according to

α^{B}(t)=R_{JtoB}R_{StoJ}α^{S}(t). (7)

After determining α^{B}, two iterations of integration and high pass filtering (2I-2HPF) at 0.3 Hz can be applied to eliminate drift and calculate an estimate of sensor displacement over time, d^{B}(t). The 2I-2HPF procedure assumes zero mean velocity and displacement of the IMU over time, which is a valid assumption for single limb and double limb standing tasks in which small oscillatory motions are observed over a fixed, base of support.

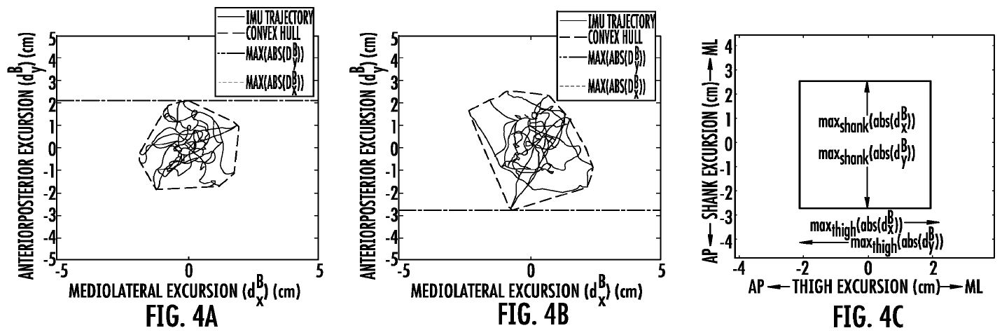

The segment excursions of the thigh and shank are produced by scatter plotting dₓ^{B} versus d_{y}^{B}, as shown in FIGS. 4A-C, from the proximal thigh and distal shank IMUs, respectively. The computation of excursion area is a traditional and widely used method in laboratory settings due to its intuitiveness for quantifying postural sway and its high test-retest reliability (0.96 ICC). A commonly used method for computing a convex hull area. The area of the convex hull can be used to assess the balance performance of the single limb and double limb standing tasks. To calculate the novel ROLS metric, each segment's excursion is calculated within the horizontal plane, the shank IMU trajectory shown in FIG. 4A and the thigh IMU trajectory shown in FIG. 4B. These maximum excursions are then combined into an excursion square area, and reported as the ROLS value, as shown in FIG. 4C. As the single limb standing involves greater excursion of the center of mass, this task is helpful for determining the reliability and validity of the ROLS metric.

To determine the reliability of the ROLS the values of the left and the right SLS can be determined twice over a 48 hour period. For example, a test pool with 20 healthy young adults (10 males, 10 females, age (years): 24.6±2.9 (22-34) (mean±standard deviation (range)), height (cm): 173.1±8.1 (154.9-186.7), weight (kg): 71.4±11.6 (60.3-97.5), could be selected. The participants can stand facing a 15 cm cone placed on the floor in front of them. Participants can be asked to cross their arms over their chest and raise one foot above the cone. The participant can then balance in this position for as long as they can, or until they reached 30 seconds. The time can be stopped if the participant's foot touch the floor, their foot is not maintained above the cone or box, their arms become uncrossed, their stance foot loses contact with the floor (i.e., hopping), or if they achieve the 30 seconds. Participants can be given a 30 second rest period between the two trials. All testers can utilize a mobile tablet with a mobile application to collect the data. Trials and testing session data can be stored automatically within the device and uploaded to a secure server. The intraclass correlation coefficient (ICC) can then be calculated to determine the test-retest reliability of the ROLS metric. In one test, the 95 percent confidence interval (CI) was determined for all ICC values. The ICC for the left and right ROLS metric was 0.99 (95% CI: 0.96-0.99) and 0.98 (95% CI: 0.94-0.99), respectively.

Methods for calculating, quantifying, and displaying ROLS can include one, or more of the following, a ROLS Symmetry Index (SI), a ROLS Injury Risk Index (IRI), a ROLS Excursion Diagram (ED), a ROLS Excursion Profile (EP), a ROLS Mean Excursion Diagram (MED), a ROLS Post-Concussive Excursion Profile (PCEP), a ROLS Post-Concussive Excursion Index (PCEI), and/or an Activity Clearance Index (ACI).

The ROLS Symmetry Index (SI) can be used to quantify SLS differences between left and right lower limbs. As previously described, the ROLS metric determines segment excursions of the thigh and shank is expressed as excursion area to quantify the stability of the lower-limb segments (i.e. thigh and shank) during static balance activities of SLS. Because people have different SLS balance strategies, there is a broad range of ROLS values has been observed. In a group of 20 young, healthy adult males, the range of ROLS values was 6.3 to 106 cm² with a mean 36 cm². While the range between subjects was great, the difference between the subjects' left and right limbs were small. Therefore, to compare ROLS values between people would not be beneficial since the differences could be too great because of age, fitness level, and body weight. Even within a given sport, difference exists between player positions, for example, in the sport of football differences have been observed between positions where larger linemen have very different balance strategies and ROLS values than the smaller wide receivers. In theory, because of body type, positional requirements, and training, the players in various positions have adopted different balance strategies. Ultimately people have different balance strategies to remain upright that produce large variances between people, to normalize these values would diminish the information that could be obtained when examining individual balance characteristics; as a result, an internal control was developed.

To generate a single value and to determine differences within each individual, a comparison of ROLS value calculated between the right and left lower limb SLS, this metric is the ROLS SI. The ROLS SI value is the symmetry between lower limbs expressed as a percentage of stability, where 100 percent suggests absolute symmetry or relatively identical ROLS values between lower limbs, and any value less than 100 percent suggests an imbalance. The lower limb with the greater ROLS value is identified as the limb with greater instability.

ROLS SI can be expressed as a percent and is determined by absolute (ABS) L (left ROLS value (cm²)) minus R (right ROLS value (cm²)) divided by L plus R and multiplied by 100.

$$R O L S Symmetry Index ( \% ) = 100 - ( {100}^{*} ABS ( \frac{L - R}{L + R} ) ) \qquad ( 8 )$$

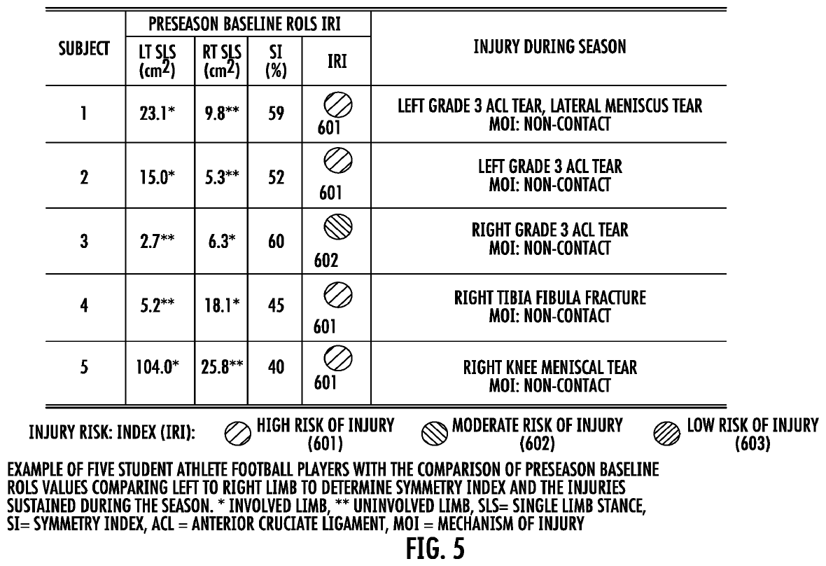

Another use for the ROLS metric is the ROLS Injury Risk Index (IM). The ROLS IRI will first be explained by way of an example study. A musculoskeletal injury prediction and determination for return to sport or activity clearance can be provided. In the table shown in FIG. 5, an example of five student athlete football players with the comparison of preseason baseline ROLS values comparing left to right limb to determine symmetry index and the injuries sustained during the season. In FIG. 5, the following annotation are explained: * Involved Limb, ** Uninvolved limb, SLS=single limb stance, SI=symmetry index, ACL=anterior cruciate ligament, MoI=mechanism of injury, and Injury Risk: Index (IRI).

The table of FIG. 5 is representative of five student athlete football players, subjects 1-5, each sustained various musculoskeletal injuries during the season. Each of the athletes during the preseason was tested for SLS and the ROLS values calculated to determine the amount of excursion of the thigh and shank segments expressed in total area (cm²). The SI can be defined as the value comparing the excursion of the thigh and shank of the left and right lower limb expressed as a percentage of symmetry between limbs. The involved, or injured, lower limb is determined to be the limb with the greatest ROLS value, and the uninvolved is the limb with the lower value. Absolute symmetry between limbs would be a SI value of 100%, any SI value less than 100% is considered asymmetrical with the involved limb being the lower limb demonstrating the greater excursion. The Injury Risk Index (IRI) is a simple color-coding, or letter grading, system that indicates the risk of sustaining an injury where: Green (603) is a low risk of injury with 100-80% of symmetry, Yellow (602) is a moderate risk of injury with 79-60% of symmetry, and Red (601) is a high risk of injury with 59% or less of symmetry between limbs.

Players 1-4 illustrate the differences between lower limbs with the SI value expressing the percentage of asymmetry. The players 1-3 all sustained non-contact anterior cruciate ligament injuries within the limb with the greatest ROLS value or instability. Player 4 had a low SI values and while jumping during practice, sustained a tibia/fibula fracture on the less stable limb. Player 5 had bilaterally very high ROLS values with very low ROLS SI injuring the limb with relatively better ROLS value, however, the ROLS value was considerably higher than expected for an athlete. It appears that the player 5 favored right limb because it may have been more stable than the left limb, but because of the relative instability of the right limb the injury occurred to the right knee. Identifying the specific anatomical structures at fault for the increased instability or higher ROLS scores and lower SI percentage can often be determined with traditional clinical examination and differential diagnosis; however, consistently, the correlation between ROLS, SI, and injury has been observed. Moreover, the instant test is less invasive and costly than traditional clinical examination and differential diagnosis.

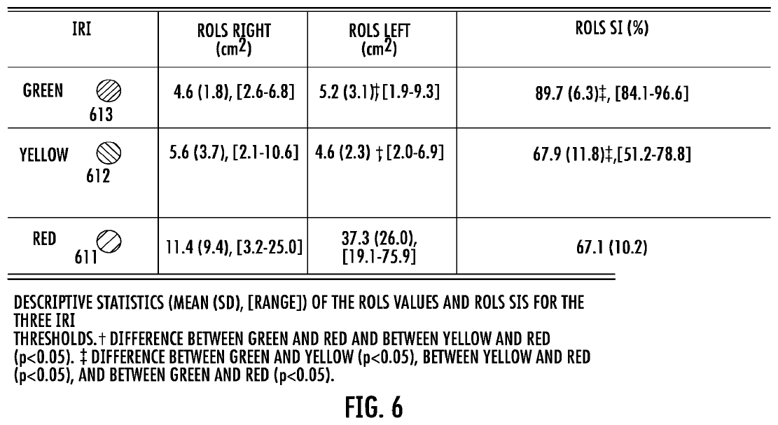

In another example, construct validation of lower limb segmental excursion as a measure of potential risk for lower limb injury in Division I women's basketball players was observed. Constructed validity of the ROLS IRI was examined in twelve Division I women's basketball players during a pre-season, in preparation for their exercise training program. The subjects were categorized based on their injury history during the season, as shown in FIG. 6: (Group 1) No reported injuries throughout the season, (Group 2) lower limb injury that did not result in missing any games, and (Group 3) lower limb injury that resulted in missing both practice and the remainder of their season. There were no differences in any anthropometric measures amongst any of the three groups. As seen in FIG. 6, no subjects classified as Green reported any injuries throughout the course of the competitive season and were identified as Group 1. Three out of four of the subjects were classified into Yellow 612 reported a history of left sided lower limb injury during the competitive season but were rehabbed conservatively and did not result in missing any games, these players were identified as Group 2. All four subjects in the Red 611 group reported lower limb injuries in a left side that resulted in missing both practice and the remainder of their season and were identified as Group 3. The study findings provided data for supporting ROLS IRI as a measure of postural stability impairment and potential risk for lower limb injury in athletes.

As shown in the table of FIG. 6, the following key may be of use: †Difference between Green (613) and Red (611) and between Yellow (612) and Red (611) (p<0.05). ‡Difference between Green (613) and Yellow (612) (p<0.05), between Yellow (612) and Red (611) (p<0.05), and between Green (613) and Red (611) (p<0.05).

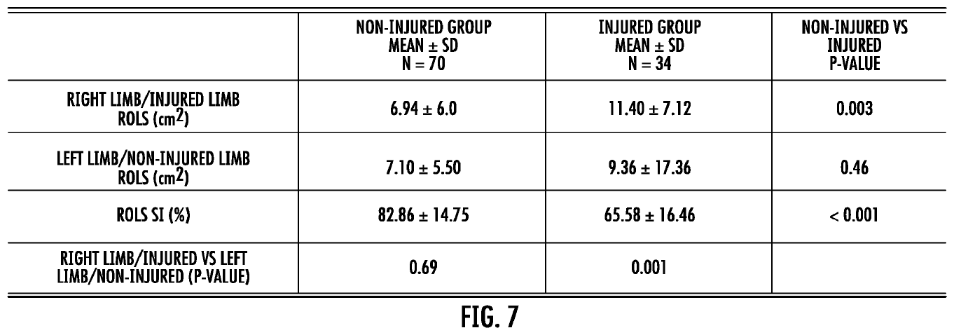

In yet another example, the accuracy of the region of limb stability in predicting risk for lower limb injury in Division I Collegiate football players is shown. In the instant example, one-hundred four Division I Collegiate Football Players participated in this study and were divided into two groups: 1) No previous lower limb injury or no in-season injury (n=70, “non-injured group”) and 2) No previous lower limb injury, but in-season injury requiring surgery (n=34, “injured group” group). The mean±standard deviation (SD) ROLS SIs was 82.86±14.75% and 65.58±16.46% for the non-injured and injured group, respectively, as shown in FIG. 7. Significant differences in ROLS SI were found between groups (p<0.001). The ROLS SI demonstrated an Area Under the Curve (AUC) of 0.8 (p<0.001; 95% CI: 0.71-0.88) with a SE of 0.04 indicating that the ROLS SI has good predictive accuracy in detecting those healthy Division I Collegiate Football Players at risk for lower limb injury resulting in surgery. The ROLS SI was found to have good predictive accuracy in detecting individuals at risk for injury that were healthy and asymptomatic during preseason testing. Increase in thigh and shank excursions and/or decrease in SI between lower limbs may be a predictor of risk for future injury.

In another example, such as with a patient that is in need of a musculoskeletal injury assessment a ROLS Excursion Diagram and a ROLS Excursion Profile may be useful. The ROLS Excursion Diagram (ED) is a graphic illustration of the excursion in the sagittal and frontal plane for either the thigh segment or the shank segment of a single limb during single limb stance. The ROLS EDs can enable a more precise examination of the trajectory, path, and total area of excursion in the two planes providing valuable insights to the patterns of excursion between segments (thigh and shank) with regards to stability, instability, and injury severity. The ROLS Excursion Profile (EP) is an outline of ROLS-ED excursion that illustrates the difference between ROLS excursion values from the A-P and M-L planes as well as the total area. In general, greater motion in one direction suggests greater instability favoring that direction, which could be related to a specific anatomical structure or a reduction in the capabilities to control motion in a particular direction by the thigh and shank.

Key elements to observe with the trajectory of excursion lines are the repeated patterns, density of the lines and where the majority of lines are found within the grid. Typically, a single rogue line is ignored because it is usually a result of a single balance correction moment, often seen early in the 30 seconds trial as the person is becoming organized to the SLS skill. Because it can be difficult to clearly visualize the patterns the ROLS Musculoskeletal Excursion Profile was created.

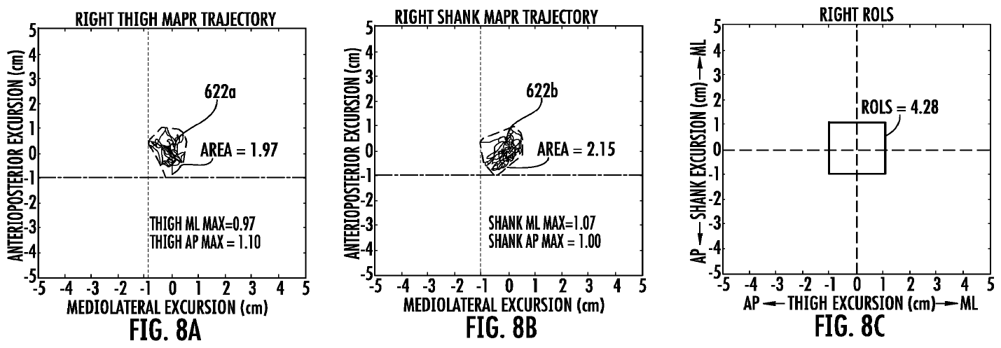

FIGS. 8A and 8B illustrate ROLS ED for the thigh and shank respectively for a first, uninjured, patient. In the illustrated figures, the excursion 622a, 622b is fairly equal in the A-P and M-L planes, with relatively a small amount of excursion (approximately 1.0 cm) with a total area of approximately 2.0 cm² for both the thigh and shank. This is representative of a person with good single limb balance, who exhibits very little sway and has good static balance strategies. In FIG. 8C, the ROLS EP illustrates an equal area when comparing excursion are between the thigh and shank in both the A-P and M-L planes suggesting again good static balance strategies without favoring any one quadrant or excessive excursion.

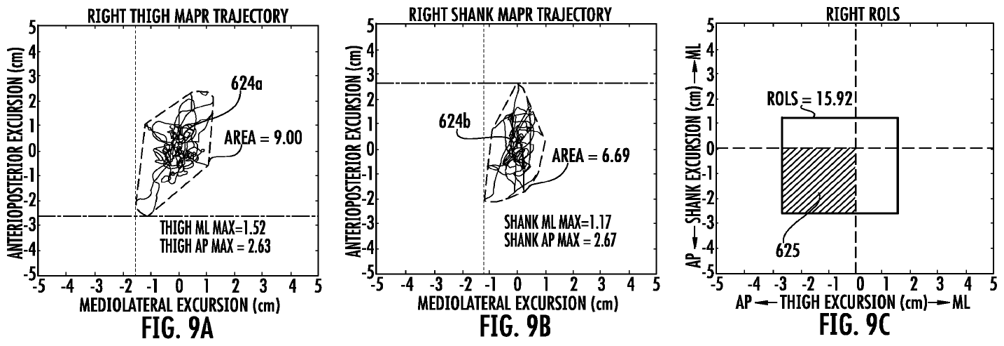

FIGS. 9A and 9B illustrate ROLS ED for the thigh and shank respectively for a second patient having a right medial collateral ligament injury. In both figures, a greater excursion 624a, 624b can be observed in the A-P plane, which is twice that of FIGS. 8A and 8B. The total area of the thigh (9.0 cm²) and shank (6.69 cm²) areas are much greater than would be expected and the thigh greater than the shank within the limb. The greater excursion is found only in the A-P plane, which could be indicative of: 1) a balance strategy pattern that has difficulty maintain stability in the A-P plane, or 2) weakness and/or injury to musculoskeletal structure(s) that control motion in the A-P plane may be at fault. In FIG. 9C, the ROLS ED provides a graphic representation of the imbalance of motion with the greatest excursion occurring in the posterior-lateral quadrant 625. Profiles such as FIG. 9C could be indicative of an athlete with a right medial collateral ligament injury.

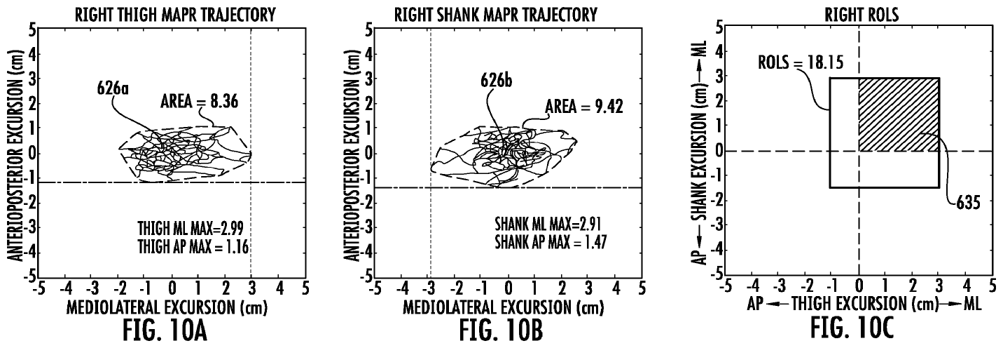

FIGS. 10A and 10B illustrates ROLS ED for the thigh and shank respectively for a third patient having a right anterior cruciate ligament injury. In both figures, greater excursion 626a, 626b can be observed in the M-L plane, almost three times that of FIGS. 8A and 8B. The total area of the thigh (8.36 cm²) and shank (9.42 cm²) areas are greater than would be expected with the thigh excursion greater than the shank within the same limb. The greater excursion is found only in the M-L plane could be indicative of: 1) a balance strategy pattern that has difficulty in the M-L plane, or 2) that weakness or injury to musculoskeletal structure(s) that control motion in this plane may be at fault. FIG. 10C shows the ROLS ED that can provide a graphic representation of the imbalance of motion with the greatest excursion occurring in the anterior-medial quadrant 635. Profiles such as FIG. 10C could be indicative an athlete with a right anterior cruciate ligament injury.

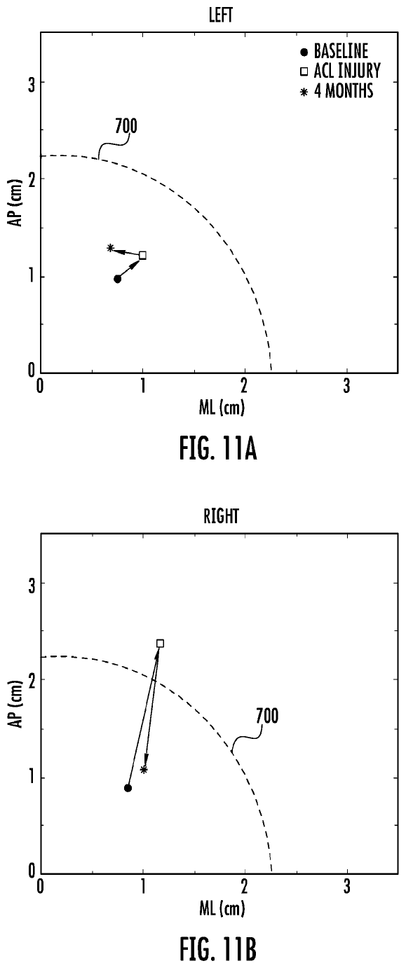

In addition to the relative area of excursion and symmetry between limbs, the ROLS can plot the mean excursion direction of displacement trajectory within and between limb in the AP and ML directions over time. In FIGS. 11A and 11B, a dotted arc line 700 can represent a baseline area that an uninvolved limb would demonstrate areas of stability for a person with a typical value range is 2-4 cm. When the mean excursion values move away from an individual's baseline area, this is representative of an SLS disturbance that can be related to musculoskeletal weakness or injury that has been sustained. The graphic display with a grid helps quantify the severity and direction of instability. The further away from baseline the mean excursion is located, the greater the instability within the lower limb or the more the severe the injury. The x-axis plots excursion in the ML (medial-lateral) direction and the y-axis plots AP (anterior-posterior) excursion. Comparisons between limbs can determine stability differences, however, the trajectory and amount of displacement can be an indication where the balance strategies are impaired, the musculoskeletal tissues at fault and the relative severity of the impairment. For example, greater displacement in the AP direction would suggest those tissues that control sagittal plane movement were at fault, while ML displacement differences may be indicative of instability in the tissues (i.e., ligament, capsule) for the frontal plane motions. Likewise, combinations of displacement may be related to altered balance strategies to compensate for the impairment. The direction of displacement and magnitude of the trajectory of the mean excursion plot has the ability to quantify the impairment and guide rehabilitative treatment. In FIG. 11A, the distance between the baseline, the ACL injury, and 4 month post injury testing are indicative of an uninjured limb. In contrast, the distance and direction of the displacement between the tests in FIG. 11B are representative of an injured limb. Of note, the legend in FIG. 11A is applicable to FIG. 11B.

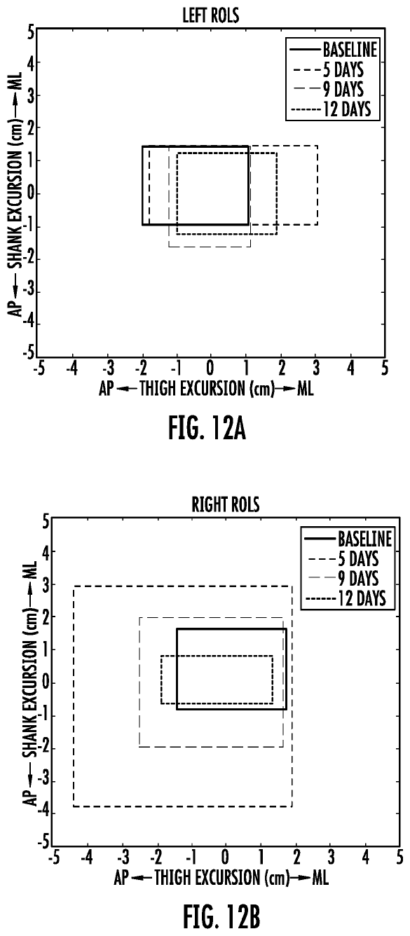

In another use, the ROLS metric can be used to determine if a patient has a concussive injury. The ROLS post-concussive excursion profile (PCEP) is a profile of ROLS excursion for the thigh and shank for a single limb in both the A-P and M-L plane illustrating the total excursion area in cm². The difference between ROLS excursion values between limbs is a measure of instability that consistent with balance issues related to a concussive injury. In general, the greater the instability or area of the PCEP, the more involved the concussive injury. As stability returns and the area of excursion decreases the more likely the concussive injury is resolving. FIGS. 12A and 12B is an example of a football player, who during a game, sustained a left-side blow to the head and subsequently was diagnosed with a concussion. The baseline test was performed prior to injury during the pre-season medical screening. His right limb is his dominate side, however in FIG. 12A, the ROLS Post-Concussive Excursion Profile (PCEP) illustrates that his SLS tests administered at 5, 9, and 12 days after injury reveals that the left limb excursion remains very similar to the baseline shape. In contrast, as shown in FIG. 12B, the right side limb PCEP excursion area is significantly increased at 5 days post-injury. The area is reduced after 9 days as other symptoms resolve and 12 days the values become slightly better than baseline, again as seen in FIG. 12B—thus indicating that the concussive injury is resolving.

Once a musculoskeletal or concussive injury has been diagnosed, an important decision is when a patient can return to sport, or other normal activity. The Symmetry Index (SI) can be defined as the value comparing the excursion of the thigh and shank of the left and right lower limb expressed as a percentage of symmetry between limbs. A concussive injury may be present when either lower limb is determined to have declined significantly after a head injury has been sustained. Absolute symmetry between limbs would be a SI value of 100%, any SI value less than 100% is considered asymmetrical with the involved limb being the lower limb demonstrating the greater excursion. The ROLS Post-Concussive Excursion Index (PCEI) is a numerical change in the area of the PCEP from a baseline measure to a subsequent measure, expressed as a percentage. The change in PCEI is typically seen after an injury or blow to the head has been sustained, but regular testing may reveal repeated smaller head injuries over time may produce positive results. As the PCEI values decline over time, the symptoms related to balance are resolving. There is no evidence to indicate that the reduction in PCEI values means that all tissue damage from the concussion has resolved but is only an indicator that balance has returned to pre-injury capabilities.

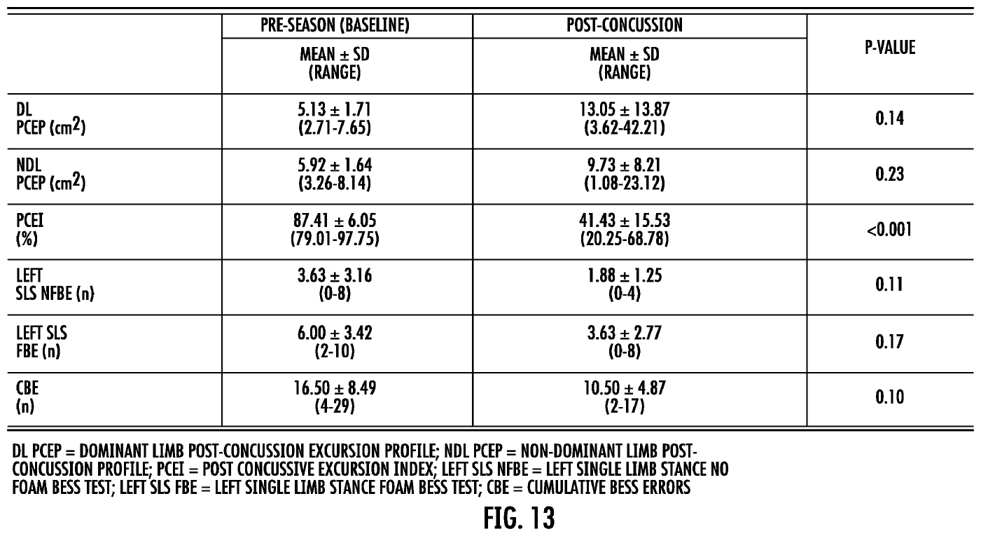

In an example, a sample of eight football players diagnosed with a concussion, were considered subjects for a case series report. All subjects underwent baseline testing prior to the start of pre-season camp. Baseline postural stability testing consisted of the SLS test and the BESS (balance error scoring system) test, respectively. Twenty-four to 72 hours following their concussion, SLS and the BESS test were administered. Segmental excursions for the thigh and shank segments for each lower limb were combined into the PCEP, which represents each segment's maximum excursion in the medial-lateral and anterior-posterior direction. The PCEI value decreased significantly post-concussion (41.43±15.53% vs. 87.41±6.05%, p<0.001) demonstrating a 52.6% decrease in inter-limb symmetry when compared to baseline values, as shown in FIG. 13. There was a 36.36% improvement in composite BESS values post-concussion (10.5±4.87 vs 16.5±8.49). Differences in inter-limb postural stability were found in post-concussion subjects. By assessing postural stability in both lower limbs individually, using the PCEI, impairments were detected that otherwise would have likely gone undiagnosed using the BESS test alone.

The Activity Clearance Index (ACI) is a simple color coding system that indicates the PCEI is closer to pre-injury values where: Green is within 80% or better of symmetry, suggesting that the athlete may be ready to return to sport games; Yellow is within 50% of symmetry, suggesting that the athlete may be ready to return to supervised practice; and Red is less than 49% of symmetry between limbs and that the athlete should not participate in practice or games. This test alone should not be used as an absolute guide for return to practice or games and a complete medical evaluation with the medical doctor's official release should be required in every case.

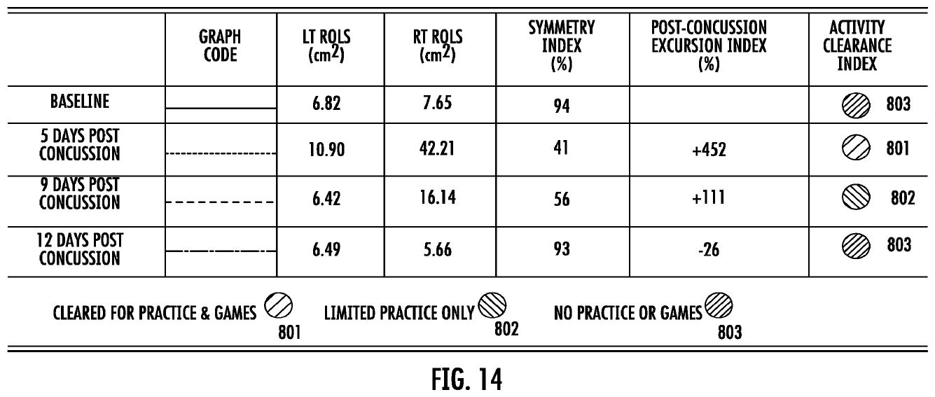

FIG. 14 is a continued example of the same football player and provides a numerical representation that corresponds with the PCEI. The ROLS values for each limb are listed for the tests administered at baseline, 5, 9 and 12 days, these values were used to determine the SI %. In FIG. 14 a green value 803 indicates the player is cleared for practice and games, a yellow value 802 indicates the player is cleared for limited practice only, and a red value 801 indicates the player is restricted from both practice and games. The corresponding PCEI values are also listed and the associated ACI. While the right side limb PCEI excursion areas are significantly different with a 452% increase just 5 days post-injury that is reduced to 111% after 9 days as other symptoms resolve and 12 days the values become slightly better than baseline. This is a table that can be provided for team medical personnel and maintained within the athlete's medical record.

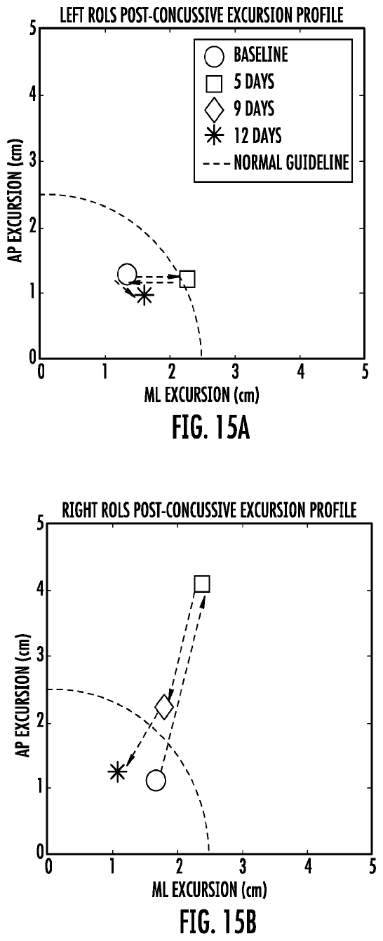

As previously described for musculoskeletal injuries, the ROLS Mean Excursion Diagram (MED) can be applied to concussion injures. The relative area of excursion and symmetry between limbs can be plotted with the mean excursion direction of displacement trajectory within and between limb in the AP and ML directions over time. As shown in FIGS. 15A and 15B, the dotted arch line represents the baseline area FOR an uninvolved limb that would demonstrate areas of stability for a person with a typical value range is 2-3 cm. When the mean excursion values move away from an individual's baseline area, there is a SLS disturbance that can be related to concussive injury. The graphic display with a grid quantifies the severity and direction of instability. The further away from baseline the mean excursion is located, the greater the instability within the lower limb or the more the severe the concussive injury. The x-axis plots excursion in the M-L direction and the y-axis plots A-P excursion. Comparisons between limbs can determine stability differences, however, the trajectory and amount of displacement can be an indication that balance strategies are impaired, a clinical sign to the extent of a concussive injury and the relative severity of the impairment.

FIGS. 15A and 15B show an example of the ROLS MED of the athlete previously described. Each point on the graph represents the mean excursion of that limb at baseline, 5, 9 and 12 days after the injury. FIG. 15A illustrates the left lower limb scores that illustrate some variance over the 12 day period but still within limits. Whereas FIG. 15B illustrates the right lower limb scores that illustrate marked deviation from baseline for days 5 and 9 and returns to baseline values on day 12. Of note, the legend shown in FIG. 15A is applicable to both FIGS. 15A and 15B.

Traditional instrumented measures of balance using IMU placed at the sacrum or waist to calculate the trajectory of the center of mass (CoM) over a both limbs during standing. Some sacral or waist systems do permit measures during SLS; however, assessment of the body CoM with traditional measures with either double limb support of SLS does not provide quantitative information about the stability within a single limb. Generally, small excursions of the CoM have been considered to be more stable than larger excursions. However, when sway is measured at the sacrum or waist the injured individuals can actually exhibit have smaller excursions of the CoM during the SLS than healthy younger persons.

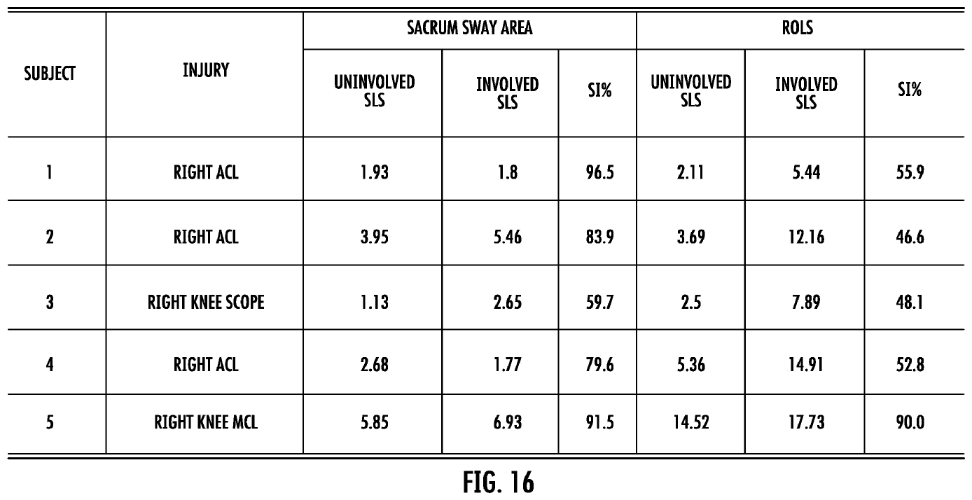

To illustrate differences between measures of balance during SLS with an IMU placed at the sacrum versus measures calculated with the ROLS, 5 injured athletes were tested while they were in the later phase of rehabilitation just prior to return-to-sport. FIG. 16 presents the excursion area values when IMUs were placed at the sacrum and above and below the knee on the lower-limb. The mean±SD SIs for the sacrum sway area and ROLS were 82.4% (±14.95) and 43.72% (±21.6), respectively with a mean difference between the two measures of 38.72 (±25.5). There are significant differences between the excursion area of the CoM when IMUs are placed on the sacrum versus each limb and the calculations based on the ROLS.

The example of five athletes with differences in SLS balance as a result of injury demonstrates that sacral IMUs cannot detect subtle changes in excursion between lower limbs that would correlate to changes in function. Therefore, current methods of instrumented SLS assessment using sacral IMUs would not be appropriate for lower limb balance and knee joint stability prior to or following a musculoskeletal injury.

In addition to the ROLS metric, there are times when use of a dynamic balance test is useful for diagnostic purposes. The quantification of dynamic balance activities and movement transition using the transitional angular displacement of segments (TADS) metric is a useful novel tool. The TADS metric is a novel measure for quantifying movements of the lower-limb segments during dynamic activity in all three planes of movement, such as: side-stepping right or left, forwards, backwards, circular, and zig-zag patterns. The TADS metric utilizes the shank sagittal angular velocity obtained with an IMU sensor to quantify the joint motion of lower limb joints during dynamic activity. Methods of measurement are similar where a set course such as the L-Test, T-Test, Illinois Test or Four Meter Side Step Test (FMSST) is performed to obtain a performance time and the TADS is employed to quaintly the movement of the lower limb. The following describes how the procedure is performed.

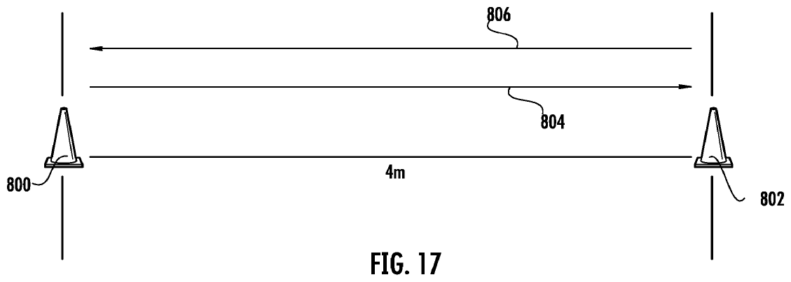

The four meter side step test (FMSST) can be performed with two cones placed four-meters apart, as seen in FIG. 17. The person being tested can start just outside of one cone 800 and can be asked to side-step as rapidly as they can, safely, towards the other cone 802 until they perform three complete passes in both directions 804, 806. Performance is measure in time to complete task. When determining the TADS four IMUs are placed, one above and one below each knee. The IMUs can be imbedded in a flexible knee sleeve, rigid knee brace or knee strap, as is the case with the SLS, as shown in FIG. 1.

The problem with using body-worn IMUs to track lower limb motions in athletes is that the artefacts caused by the soft tissue movements most likely do not accurately represent the motions of the lower limb during highly dynamic movements. Even the isometric contraction of a muscle under a sensor can create IMU movement artefact, without any movement of the knee joint. Likewise, manual placement is not easily standardized across subjects, especially in situations where subjects are required to don and remove the IMUs themselves. These problems may change the value of signal integrals and introduce error and variability in motion-related parameter estimates. While IMUs have attractive features compared to traditional laboratory-based equipment, there is no clinical-guideline-based protocol to standardize measures derived with this approach despite their widespread use among clinicians. Non-standardized kinematic measures derived from IMUs may affect the reliability or validity of instrumented clinical testing. To resolve this issue, the instant invention makes use of donned IMUs on a knee sleeve with elastic bands that keep the sensors in the medial tibial flare which is a flat surface on the anteromedial aspect of the knee just above the tibial tubercle. The orientation of IMUs (100a-c) donned on the medial tibial flare were virtually aligned (102a-d) with respect to the lateral joint line as mentioned with respect to FIG. 2. The sensor alignment process with knee sleeves allows sensors to be placed at the same locations, to be standardized across subjects, and to obtain artefact-reduced signals.

TADS is a measure that quantifies the amount of total angular displacement of the shank over time. While reference is made to the thigh and shank, for the ease of discussion, one of ordinary skill in the art will understand that TADS can additionally be used to quantify the amount of total angular displacement of any limb relative to another reference point on the subject. TADS is derived from a shank IMU during change of direction (CoD) while performing the FMSST because measures of the shank movement can be reflective of knee moments and kinematics. During the FMSST CoD period, the outside foot makes contact with the ground as the limb begins to decelerate the lateral movement of the body. Also, high loading conditions can occur in segments around the knee joint, all of which require movements in multiple planes. Though the basic movements of the shank are flexion and extension in the sagittal plane, abduction loads and movements in the frontal plane should be also considered to identify the multi-planar mechanism of the shank segment movements during the CoD period. Therefore, an understanding of the difference between the coupled shank segment movements during the FMSST CoD periods may help to determine distinct movement strategies and identify movement impairments due to a knee ligament injury.

A vector coding method has been commonly used to quantify intersegmental coordination variability. In the instant system and method, for each instant i during the FMSST test, quantification of interplanar coupled angle, vᵢ in (9), is obtained using a modification of a vector coding technique and calculated as follows:

$${v}_{i} = {tan}^{- 1} ( \frac{{y}_{i + 1} - {y}_{i}}{{x}_{i + 1} - {x}_{i}} ) , where i = 1 , 2 , … , n . \qquad ( 9 )$$

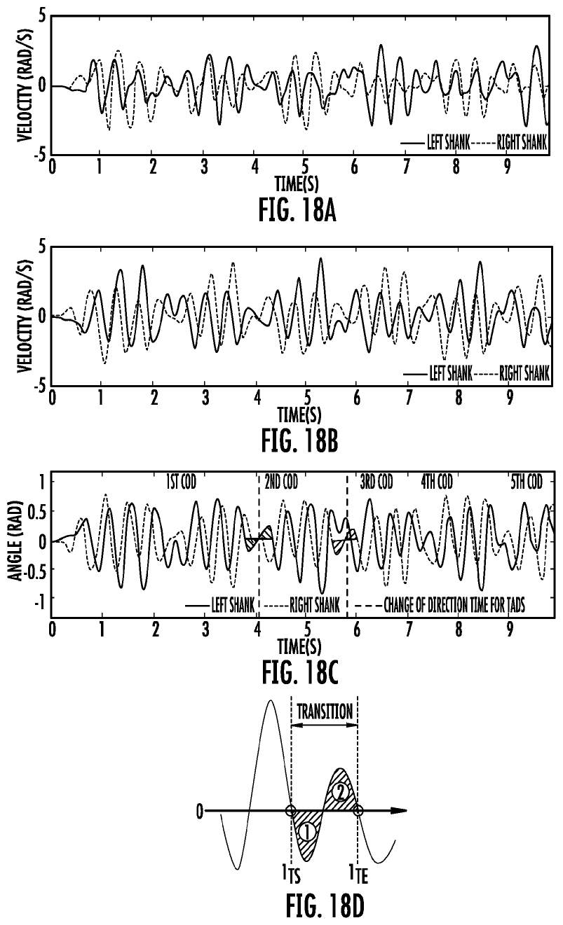

The values of x and y reference the shank angular velocities of the sagittal plane and frontal plane, respectively. The sagittal and frontal angular velocities correspond to the angular velocity around the M-L and A-P axes of the knee joint, respectively. As shown in FIGS. 18A and 18B, the red dotted and blue solid waves represent the angular velocities recorded from the right and left shank IMUs, respectively. By using the angular velocities, the coupled angular displacements (in rad) of the right shank and left shank were calculated as represented in the wave diagram of v, (9), illustrated with the red dotted and blue solid waves in FIG. 18C. As shown in the enlarged view, FIG. 18D, knee flexion and extension during the FMSST CoD were matched to negative displacement (\textcircled{1}) and positive displacement (\textcircled{2}) based on the aligned orientation. The negative displacement (\textcircled{1}) and positive displacement (\textcircled{2}) are graphically depicted with a subject 1000 in FIGS. 18E and 18F, respectively.

The TADS metric is based on a concept of the area under the wave at each CoD period (shaded areas in rad×s), resulting in the integral of angular displacement from the transition start (t_{TS}) to the transition end (t_{TE}) (FIG. 18D). The event signal to identify transition time durations is automatically generated by detecting the CoD using a knee IMU or a pelvic IMU, and a computer application. Specifically, the acceleration data recorded from the pelvic IMU was smoothed with a Butterworth low-pass filter at a normalized frequency of 0.1, resulting in a distinct sinusoidal relationship between trial time and M-L axis acceleration. The peaks and troughs of this sinusoid represented moments of zero velocities and were used to detect changes in speed and direction of motion. Five transitions (vertical black and gray dotted lines in FIG. 18C) were flagged during one trial of the FMSST. Two middle transitions (two vertical black dotted lines in FIG. 18C) the second and third transitions for TADS Left and TADS Right, respectively) were used among five transitions with 180° of change in direction. The first transition (CoD from right to left) was discarded in order to exclude the data of the transition from standing to running.

The reliability of the TADS test can be explained by way of concurrent testing of 20 subjects, who participated SLS tests for the ROLS reliability and performed the FMSST with TADS calculated for the shank. After donning sleeve having the IMUs, the FMSST were performed on an indoor gymnasium with wood flooring. All subjects completed two trials of the FMSST. They were given a 60 second rest period between each trial. For the test-retest reliability study, subjects were evaluated twice within a 48-hour period, under identical testing conditions. The best/fastest trial of the FMSST was used for intraclass correlation coefficient data analysis for all subjects. For example, the fastest FMSST trial performed by each subject was used on day 1 and day 2 to assess test-retest intraclass correlation coefficient reliability of the TADS metric. The total time taken to complete the FMSST as fast and as safely possible was also recorded by a tester via a computer application.

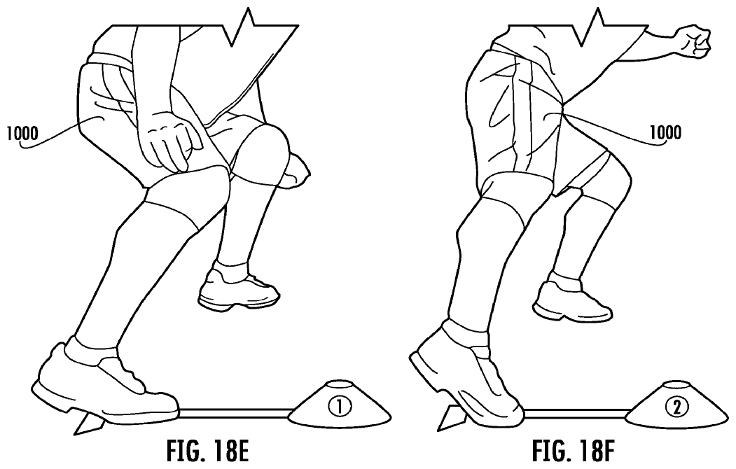

As shown in FIG. 19, the intraclass correlation coefficient for test-retest reliability for FMSST, TADS right lower limb, TADS left lower limb, and TADS symmetry index was 0.90 (95% confidence interval: [0.61-0.95]), 0.87 [0.63-0.96], 0.89 [0.64-0.96], and 0.81 [0.58-0.92], respectively for the 20 healthy subjects. Thus, the TADS is a reliable measure of dynamic balance and movement transition.

There are various methods for calculating, quantifying, and displaying TADS metrics. Those methods include, but are not limited to, 1) TADS Transition Index (TI); 2) TADS Symmetry Index (SI); 3) TADS Injury Risk Index (IRI); 4) Activity Clearance Index (ACI); and 5) Static-Balance: Dynamic-Agility (SBDA) Index.

In a first example, the TADS transition index (TI) can make use of the high speed transition measurements of the FMSST. The FMSST was instrumented, as it is a commonly used performance-based outcome measure to assess agility. While high speed transitions and cutting movements are frequently performed by athletes on the field, limited information is available on the relationship between knee function and shank angular velocity during these movements. The TADS Transition Index (TI) can be computed during the transition periods of the FMSST when the outer limb makes contact with the ground in order to decelerate the lateral movement of the body in one direction. The outside limb's hip, knee, and ankle are in a flexed position preparing for lateral acceleration in the opposite direction, which results in extension of the hip, knee, and ankle. Other tests have been used to assess knee segmental angular velocities, such as the single- and double-leg hopping. In those studies, segmental angular velocities were used in the analysis of knee flexion during activities involving rapid deceleration. Segmental angular velocities can allow for analysis of the individual movements of the thigh and shank in addition to their relative movement. Thus, segment angular velocities of the lower limb are important for understanding lower extremity kinematics. In a group of 20 young healthy adults the range of TADS TIs was 10 to 73 degree with a mean 42 degree. Therefore, similar to the ROLS, to compare TADS TIs between people would not be beneficial since the differences could be too great, because of age, fitness level, previous sport, and body weight. Additionally, since shank flexion/extension range of motion can be limited, difference of TADS TIs of both legs could be an additional metric to assess dynamic agility deficits and outcomes.

TADS TI can be used for performance enhancement and injury prevention. For example, a TADS TI can indicate asymmetry between limbs such as the knees. If an athlete has sustained a knee ligament injury, they often have altered and compensatory movement patterns. Also, CoD maneuvers in athletes may be different between their sports, gender, or even the different player positions within a sport. Therefore, determining any interlimb symmetry may aid in developing a more thorough understanding of variability in segmental motion between and within sports. As a result, symmetry between limbs of an individual athlete (individual) is the best control for the TADS or obtaining a preseason baseline test for post injury comparison. Like ROLS SI, TADS SI is expressed as a percent and is determined by absolute (ABS) L (left TADS TI (degree)) minus R (right TADS TI (degree)) divided by L plus R and multiplied by 100.

$$TADS Symmetry Index ( \% ) = 100 - ( {100}^{*} ABS ( \frac{L - R}{L + R} ) ) \qquad ( 10 )$$

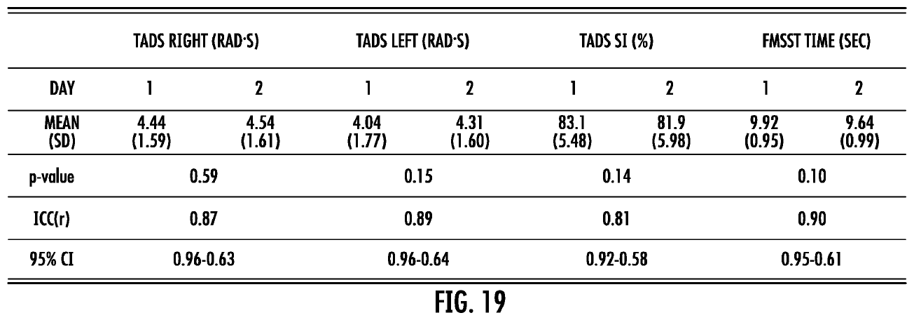

In an example, as illustrated in FIG. 20, a table is shown describing the metrics for two student football players who sustained musculoskeletal lower injuries during the season and one with no season injury. Each of the athletes during preseason was tested for FMSST and the TADS TIs calculated to determine the amount of flexion and extension of the shank segment expressed in total degree. The SI (symmetry index) is the value comparing the TADS TIs of the left and right lower limb expressed as a percentage of symmetry between limbs. The involved lower limb is determined to be the limb with the smallest TADS TI and the uninvolved is the limb with the greater TADS TI value. Absolute symmetry between limbs would be a SI value of 100% and any SI value less than 100% is considered asymmetrical with the involved limb being the lower limb demonstrating the smaller TADS TI. The Injury Risk Index (IRI) is a simple color coding system that indicates the risk of sustaining an injury where: Green 803 is low risk of injury with 100-80% of symmetry, Yellow 802 is moderate risk of injury with 79-60% of symmetry, and Red 801 is high risk of injury with 59% or less of symmetry between limbs.

Subjects 1 and 2, of FIG. 20, illustrate the differences between lower limbs with the SI value expressing the percentage of asymmetry. The subjects 1 and 2 each sustained non-contact anterior cruciate ligament injuries within the limb with the smallest TADS TI. Identifying the specific anatomical structures at fault for the limited movement or lower TADS TI/SI percentage can often be determined with traditional clinical examination and differential diagnosis.

In addition to the TADS TI, the Activity Clearance Index (ACI) is a simple color coding system that indicates the TADS SI is closer to pre-injury values where: Green 903 is within 80% or better of symmetry, suggesting that the athlete may be ready to return to sport games; Yellow 902 is within 50% of symmetry, suggesting that the athlete may be ready to return to supervised practice; and Red 901 is less than 49% of symmetry between limbs and that the athlete should not participate in practice or games. This test alone should not be used as an absolute guide for return to practice or games and a complete medical evaluation with the medical doctor's official release should be required.

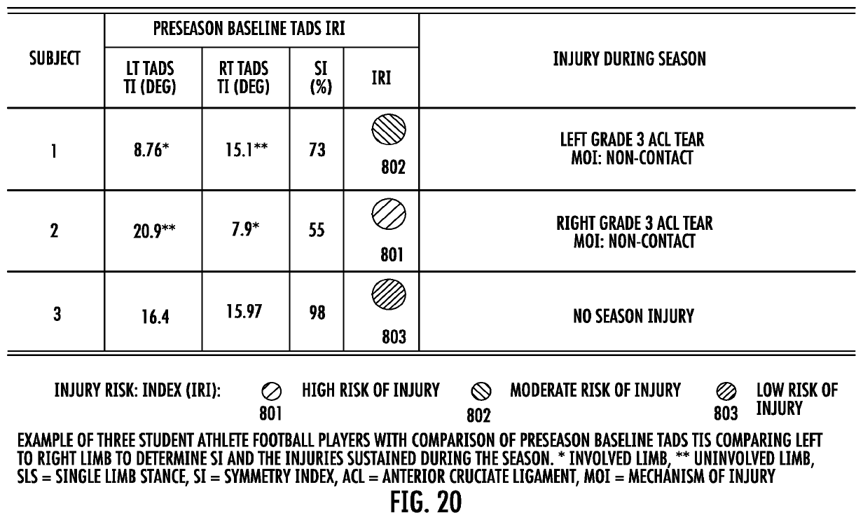

Further, in combination with the ROLS metric, a dynamic mobility test, the FMSST, can stress athletes safely while allowing for assessment of agility, posture, and coordination, and should be administered at time-points where static balance testing no longer indicates injury. As shown in FIG. 21, 5 month post ACL injury, the activity clearance index (ACI) of ROLS has returned to green 903. However, the SI between left and right TADS TIs was still 73%, thus the ACI is yellow 902, indicating the need for additional rehabilitation.

Twelve days post-concussion, the ACI of ROLS also returned to green 903; however, at the time-point in which static balance testing was no longer indicative of concussion, the SI between left and right TADS TIs was still 71% (the ACI is also yellow), indicating the need for additional rehabilitation. Thus, combination of static and dynamic symmetry could be an additional metric to assess overall deficits and outcomes. The Static-Balance: Dynamic-Agility (SBDA) Index is expressed as an equation for combining static and dynamic SIs as follows:

$$Root Mean Square ( RMS ) = \sqrt{\frac{( {Static}^{2} + {Dynamic}^{2} )}{2}} \qquad ( 11 )$$

where ‘Static’ and ‘Dynamic’ in equation (11) are ROLS SI and TADS SI, respectively.

In an example, it can be beneficial for the quantification of a novel agility testing metrics with inertial sensors following knee injury in Division I collegiate athletes. In a test group of 200 university athletes, individual baseline testing was conducted. Among the athletes, fifteen (1 female tennis, 2 female basketball, and 12 football) who sustained a knee ligament injury were tested again at the time of return to sport (RTS). The athletes passed commonly used tests prior to RTS including clearance by an orthopaedic surgeon, multi-speed isokinetic dynamometry testing, and unilateral assessments of power and endurance.

All of the 200 subjects completed two trials of the FMSST with the instant system. They were given a 60 second rest period between each trial. They were given a third trial to complete if they were disqualified from one of the first two trials. A subject is deemed to be disqualified if: 1) they fail to touch or cross the left or right outside tape mark; 2) they fail to keep their trunk and feet pointing forward at all times; and/or 3) they cross their legs. The total time taken to complete the FMSST as fast and as safely possible was also recorded by a tester via the instant application. Athletes were also tested twice during the pre-season and at the time of RTS after injury.

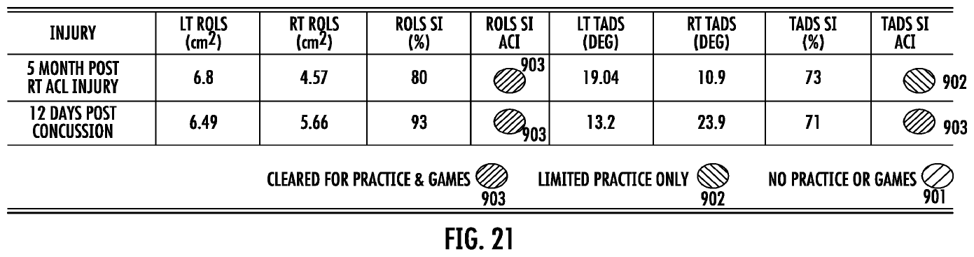

For subjects with a previous knee ligament injury (n=15), the baseline and RTS FMSST times were mean 9.26 (SD 1.31) and mean 9.79 (SD 1.99), respectively as seen in FIG. 22. Each TADS SI mean and SD is also presented in FIG. 7. Specifically, these subjects demonstrated a mean 85.82 (SD 4.84) between the injured and non-injured limbs at the baseline test. TADS SI at the time of RTS testing was mean 76.44 (SD 13.22) between the injured and non-injured limbs. Statistical significance was achieved when comparing the TADS SI of the baseline test to the RTS test (P=0.046). However, comparisons between the baseline and RTS test in FMSST time did not reach statistical significance (P=0.32) (See Table 11). Lastly, the effect size was calculated from the change in TADS SI and FMSST time from baseline to RTS. A large effect size (d=−1.04) was observed in the change in TADS SI from baseline to RTS. A small to medium (d=0.32) effect size was observed in the change FMSST time from baseline to RTS.

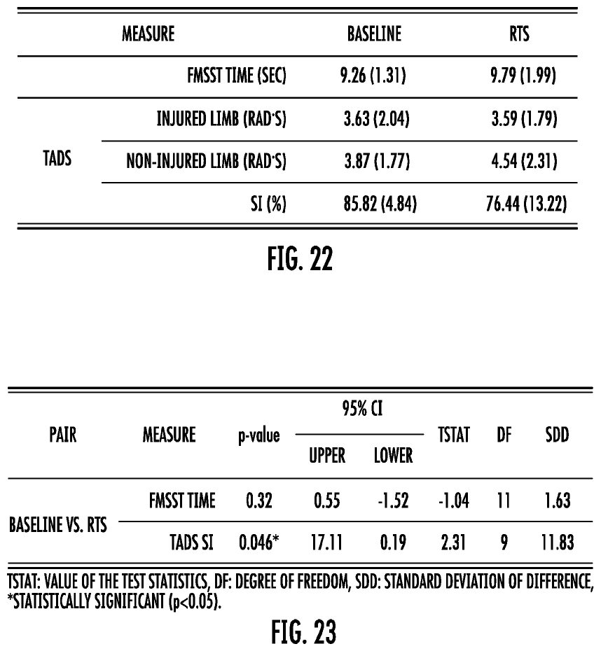

Knee ligament re-injury can occur as a result of asymmetrical loading patterns or altered biomechanics. Many re-injuries to the knee have been attributed to altered biomechanics between the lower limbs that persist beyond the period of rehabilitation that were not detected prior to completion of rehabilitation. For example, subjects with a history of ACL reconstruction can demonstrate an asymmetrical force distribution between limbs during dynamic tasks such as landing and squatting for up to 15 months post-surgery, which is often beyond the typical time for RTS. Even when lower limb strength and endurance are improved following rehabilitation, neuromuscular coordination can remain impaired for 18 months or more. FIG. 23 shows that the significant changes in the athletes indicating a greater limb asymmetry at the time of RTS were attributed to changes in the non-injured limb. This may be related to the training effect during the rehabilitation period. For example, movement asymmetries after ACL reconstruction may be due to athletes' preference of using their contralateral uninjured limb. The decreased coordination can also have adverse effects on the contralateral limb. Athletes with asymmetric loading of the contralateral limb may have further risk for next ACL injury because limb loading asymmetry has been considered as a potential ACL injury risk factor. Therefore, it is believed that identification of asymmetry caused by the uninjured limb could be an important component for safe reintegration of athletes back to sports. The lack of sensitivity in the FMSST time and the possibility of compensation during bilateral activities may mask unilateral deficits and increase the risk of re-injury.

The instant system and methods result in a major time saving for testing, processing, and displaying results in real-time because most sports teams and collegiate student athletes have very busy schedules, have little spare time, and want to know there results immediately. This is especially true for military personnel, clinicians, and coaches.

In one exemplary method of use, these individuals are asked to place a belt around their waist and slip on two knee sleeves. While the sensor sleeves are donned the athlete's record is found on an IPAD, or other computing device, and the IMU sensors are paired to the IPAD once the athlete stands. The athlete can then perform a walking calibration test which takes a mean of 13 seconds to complete (n=288). The SLS test takes 30 seconds per limb, with a 30 second rest between trials, for a total of 90 seconds. The mean time to complete the FMSST for all sports (n=469) was 9.5 seconds per trial with a 60 second rest between the 2 trials for a total of 80 seconds. The athlete can then remove the waist belt and takes the knee sleeves off. The total time to complete the testing according to the mobile app time stamps, from the initial encounter to exit, was approximately 4.3 minutes. The results are real-time and can be located on the IPAD immediately after testing on a summary page. The less than 5-minute testing time and real-time feedback can translate into substantial time savings over the life of military cadets and collegiate student athletes. By detecting potential functional impairments and reducing re-injuries, the time savings over a year to a military academy and collegiate athletic program could far outweigh the few minutes of testing. The savings in medical costs could be substantial as well. The novel IMU-based metrics within the instant system is a valid and reliable system for the evaluation of different populations is practical to apply and provides real-time results.

It would be appreciated by those skilled in the art that various changes and modifications can be made to the illustrated embodiments without departing from the spirit of the present invention. All such modifications and changes are intended to be covered by the appended claims.

What is claimed is:

1. A method for asssessong, monitoring, or theranosing a condition or disorder based on a comparison of limb stability for one or more limbs of a subject from a baseline, the method comprising:
placing two or more inertial measurement sensors on a left limb of the subject and two or more inertial measurement sensors on the corresponding right limb of the subject;
acquiring baseline limb excursion data from the inertial measurement sensors while a patient is performing at least one of a static balance activity by tracking the relative displacement of the respective two or more inertial measurement sensors and a dynamic balance activity by tracking the relative displacement of the respective two or more inertial measurement sensors;
acquiring post-injury limb excursion data after an injury from the inertial measurement sensors while a patient is performing at least one of a static balance activity by tracking the relative displacement of the respective two or more inertial measurement sensors and a dynamic balance activity by tracking the relative displacement of the respective two or more inertial measurement sensors; and
determining a theranosis or a condition as a function of a comparison of the baseline limb excursion data compared to the post-injury limb excursion data.

2. The method of claim 1, wherein the post-injury limb excursion data is a region of limb stability for the respective limb and is calculated by each segment's medio-lateral and anterior-posterior excursions within the horizontal plane and combining the maximum excursions into an excursion square area for each of the left and right limbs.

3. The method of claim 2, wherein the determining step is calculated as function of
$$a Symmetry Index ( \% ) = 100 - ( {100}^{*} ABS ( \frac{L - R}{L + R} ) ) ,$$
where L is the left limb region of limb stability value (cm²), and
where R is the right limb region of limb stability value (cm²).

4. The method of claim 3, further comprising,
determining one of,
if the Symmetry Index is greater than a first threshold value allowing the subject to return to regular activities;
if the Symmetry Index is less than the first threshold value and more than or equal to a second threshold value allowing the subject to return to supervised activities, or
if the Symmetry Index is less than the second threshold value restricting the subject from participating in regular activities,
wherein the first threshold value is greater than the second threshold value.

5. The method of claim 1 further comprising
a processing alignment procedure to establish and capture a rotation matrix of the subject performing a stand-still step and a linear gate step,
calculating a known relationship for the processing alignment of one of the inertial measurement sensors roll, pitch, and yaw as a function of a fixed time-invariant rotation from one of the inertial measurement sensors frame of reference from a limb joint frame of reference.

6. The method of claim 1 further comprising a two-phase signal processing alignment procedure to establish a known relationship between the four relevant reference frames: the sensor frame S=<Sₓ, S_{y}, S_{z}>, the joint frame J=<Jₓ, J_{y}, J_{z}>, the body frame B=<Bₓ, B_{y}, B_{z}>, and the global frame G=<Gₓ, G_{y}, G_{z}>.

7. The method of claim 1, where the post-injury limb excursion data is calculated by:
obtaining the shank sagittal angular velocity data for one or more shanks of the subject during a predefined activity with an inertial measurement sensor placed on the shank;
determine ω_{ML}^{B}(t_{TS}) and ω_{ML}^{B}(t_{TE}) which are the times for transition start and transition end for a change indirection of movement of the subject, respectively on a graph of the angular velocity data ω^{B}(t); and
calculate the area under the curve by integrating a line joining ω_{ML}^{B}(t_{TS}) and ω_{ML}^{B}(t_{TE}) at all transitions.

8. The method of claim 7 further comprising combining the sagittal angular velocity of shank with the sagittal plane angular velocity of the thigh and the coronal plane velocities to quantify motion of the knee joint.

9. The method of claim 6, wherein the determining step is calculated as function of
$$a Symmetry Index ( \% ) = 100 - ( {100}^{*} ABS ( \frac{L - R}{L + R} ) ) ,$$
where L is the left area under the curve value (degree), and
where R is the right area under the curve value (degree).

10. The method of claim 9, further comprising,
determining one of,
if the Symmetry Index is greater than a first threshold value allowing the subject to return to regular activities;
if the Symmetry Index is less than the first threshold value and more than or equal to a second threshold value allowing the subject to return to supervised activities, or
if the Symmetry Index is less than the second threshold value restricting the subject from participating in regular activities,
wherein the first threshold value is greater than the second threshold value.

11. The method of claim 1, wherein the injury or condition is a concussion.

12. The method of claim 1, wherein the two or more inertial measurement sensors on the left limb of the subject are fixed to a left limb sleeve and two or more inertial measurement sensors on the right limb are fixed to a right limb sleeve.

13. The method of claim 1,
wherein two or more inertial measurement sensors on the left limb are placed below the knee joint line at the medial tibial flare on a left shank of the subject and above a left knee joint line, along with the distal iliotibial band on a left thigh of the subject, and
wherein two or more inertial measurement sensors on the right limb are placed below the knee joint line at the medial tibial flare on a right shank of the subject and above a right knee joint line, along with the distal iliotibial band on a right thigh of the subject.

14. The method of claim 1, further comprising placing an additional inertial measurement sensor at the center of the sacrum of the subject.

15. The method of claim 14, wherein the additional inertial measurement sensor confirms a change in direction of the subject.
